(19) United States
(12) Patent Application Publication
(10) Pub. No.: US 2011/0224249 A1
(43) Pub. Date: Sep. 15, 2011

(54) Novel Hydrate Form

(75) Inventor: Axel Becker, Darmstadt (DE)

(73) Assignee: Generics [UK] Limited, Potters Bar (GB)

(21) Appl. No.: 13/111,799

(22) Filed: May 19, 2011

Related U.S. Application Data
(62) Division of application No. 12/339,196, filed on Dec. 19, 2008, now abandoned.

(30) Foreign Application Priority Data
Jun. 23, 2006 (GB) 0612422.6
Jun. 22, 2007 (GB) PCT/GB2007/050349

(51) Int. Cl. A61K 31/4709; C07D 471/04; A61P 31/00; A61P 31/04

(52) U.S. Cl. 514/300; 546/113

(57) ABSTRACT

The present invention relates to a novel hydrate form of moxifloxacin monohydrochloride, processes for preparing the form, pharmaceutical compositions comprising the form and uses of the form and compositions.

### CROSS REFERENCE TO RELATED APPLICATIONS

This application is a continuation of International Patent Application No. PCT/GB2007/050349, filed on Jun. 22, 2007, which claims priority to Great Britain Application No. 0612422.6, filed on Jun. 23, 2006, the entire contents of both of which are incorporated herein by reference.

### TECHNICAL FIELD

The present invention relates to a novel hydrate form of the monohydrochloride salt of the antibacterial drug moxifloxacin, 1-cyclopropyl-7-([S,S]-2,8-diazabicyclo-[4.3.0]non-8-yl)-6-fluoro-1,4-dihydro-8-methoxy-4-oxo-3-quinolinecarboxylic acid, shown below. The present invention further relates to processes for preparing the form, pharmaceutical compositions comprising the form and uses of the form and compositions. The pharmaceutical compositions may be used, in particular for the treatment of bacterial and microbial infections.

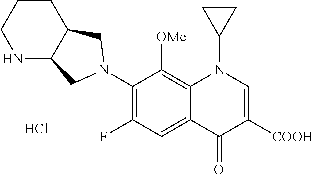

### BACKGROUND ART

The manufacturing process for many pharmaceuticals is hindered by the fact that the organic compound which is the active drug substance has an irregular or unstable crystalline form. In some cases, such irregularities can cause handling difficulties during the manufacturing process and/or undesirable properties being imparted to the final drug or dosage form. The latter include inconsistent drug substance dissolution rates and the like.

In addition, some crystalline or amorphous forms are thermodynamically unstable and may convert to more stable forms during manufacturing and/or during storage. This interconversion can cause inconsistencies in dissolution rate and bioavailability which is unacceptable for the approval of a marketed pharmaceutical.

Moxifloxacin and its addition salts were first disclosed in U.S. patents U.S. Pat. No. 4990517 and U.S. Pat. No. 5607942 and moxifloxacin monohydrochloride is currently marketed as a broad spectrum antibacterial agent.

Crystalline polymorphic forms of moxifloxacin monohydrochloride have been described in U.S. patent U.S. Pat. No. 5849752 (anhydrous Form I and monohydrate Form II) and in international patent application WO 04/091619 A1 (anhydrous Form III). An amorphous form of moxifloxacin monohydrochloride has been disclosed in international patent application WO 04/039804 A1.

It has now been surprisingly found that moxifloxacin monohydrochloride has a hydrated form which is more stable than the monohydrate form reported in U.S. patent U.S. Pat. No. 5849752. This novel hydrate form will be more stable during the shelf life of the product and consequently the novel form of the present invention will be suitable to use as a pharmaceutical and have the advantages over other crystalline or amorphous forms described earlier.

### SUMMARY OF THE INVENTION

It is an object of the present invention to provide moxifloxacin monohydrochloride in a stable hydrated form that affords the compound improved handling properties and/or improved properties as a pharmaceutical agent.

Therefore, a first aspect of the present invention provides a hydrate form of moxifloxacin monohydrochloride having an X-ray diffraction pattern comprising at least three peaks selected from peaks with 2 theta angles of about 5.8, 7.2, 8.6, 10.3, 17.3, 17.9, 19.3, 21.6 and 27.4 degrees, when Cu Kα radiation (λ=1.5406 Å) is used. Preferably, the hydrate form of moxifloxacin monohydrochloride has an X-ray diffraction pattern comprising at least four, five, six, seven, eight or nine peaks selected from peaks with 2 theta angles of about 5.8, 7.2, 8.6, 10.3, 17.3, 17.9, 19.3, 21.6 and 27.4 degrees, when Cu Kα radiation (λ=1.5406 Å) is used. Preferably, the peaks are selected from peaks with 2 theta angles of about 5.8, 7.2, 8.6, 10.3, 17.9, 19.3, 21.6 and 27.4 degrees.

The first aspect of the present invention also provides a hydrate form of moxifloxacin monohydrochloride having an X-ray diffraction pattern substantially as shown in FIG. 1, when Cu Kα radiation (λ=1.5406 Å) is used.

Slight variations in the observed 2 theta angles are expected based on the specific diffractometer used, the analyst and the sample preparation technique. The terms ‘2 theta angles of about’ and ‘an X-ray diffraction pattern substantially as shown’ are to be interpreted accordingly.

Preferably, the hydrate form of the first aspect of the present invention is substantially free of other polymorphic and amorphous forms of moxifloxacin monohydrochloride. This means that the hydrate form of the present invention comprises less than 10% of other polymorphic and amorphous forms, preferably less than 5%, preferably less than 1%.

The hydrate form in accordance with the invention can be used to advantage in the preparation of pharmaceutical dosage or drug forms. When in particulate form, the hydrate form in accordance with the invention is stable and free flowing and does not present any of the stability (e.g. polymorphic conversion or chemical conversion) or handling difficulties associated with other forms of crystalline or amorphous moxifloxacin monohydrochloride. The novel hydrate form according to the invention, therefore, can be employed in the manufacture of pharmaceutical compositions that do not suffer from the problems, such as inconsistent drug substance dissolution rates and the like, that can be manifest in dosage forms manufactured using previously available forms of moxifloxacin monohydrochloride.

A second aspect of the present invention provides a process for the preparation of the hydrate form of moxifloxacin monohydrochloride of the first aspect of the invention, comprising humidification of one or more forms of moxifloxacin monohydrochloride. The forms of moxifloxacin monohydrochloride used may be amorphous moxifloxacin hydrochloride or one or more crystalline forms of moxifloxacin hydrochloride or a mixture thereof. Preferably, the humidification is controlled humidification.

Preferably, the humidification is carried out at 50-90% relative humidity at 25-60° C. for 8-24 hours. Preferably, the reaction is carried out at a relative humidity of 60-90%, preferably 60-80%. Preferably, the reaction temperature is in the range of 30-60° C., preferably 30-50° C., preferably 30-40° C. Preferably, the reaction is carried out over 10-22 hours, preferably 12-18 hours.

Preferably, the humidification is carried out at about 60% relative humidity at about 30° C. for about 18 hours. Alternatively, the humidification may be carried out at about 80% relative humidity at about 30° C. for about 12 hours.

Any instrument which monitors and controls humidity may be used for the humidification process of the present invention. Hence, the humidification can be carried out in a humidity or stability cabinet (such as a Binder KBF® climatic chamber) or in the humidity chamber of an analytical instrument (such as the humidity chamber of an X-ray diffractometer or a Gravimetric Vapour Sorption instrument).

In further aspects, the present invention provides a method of preparing a pharmaceutical dosage form that utilises the hydrate form in accordance with the first aspect of the invention. It also provides a pharmaceutical dosage form prepared or preparable by such a method. The dosage form can be a solution or suspension form, but is preferably solid and comprises one or more conventional pharmaceutically acceptable excipient(s). Preferred dosage forms in accordance with the invention include tablets, capsules and the like. Tablets can be prepared by conventional techniques, including direct compression, wet granulation and dry granulation. Capsules are generally formed from a gelatine material and can include a conventionally prepared granulate of excipients and adduct or solvate in accordance with the invention. Preferably, the dosage form is for oral administration.

The hydrate form in accordance with the first aspect of the invention may also be useful as precursor to other novel or known polymorphic forms of moxifloxacin monohydrochloride that may be useful in the preparation of pharmaceutical products.

In a further aspect of the invention, there is provided the use of the hydrate form of the first aspect of the invention, in the preparation of a medicament for the treatment of a bacterial or microbial infection. Also provided is a method of treating a bacterial or microbial infection, comprising administering a therapeutically effective amount of the hydrate form of the first aspect of the invention to a patient in need thereof. Preferably, the patient is a mammal, preferably a human.

The present invention is illustrated but in no way limited by the following examples and figure.

### BRIEF DESCRIPTION OF FIGURES

FIG. 1 shows the XRPD pattern of the novel moxifloxacin monohydrochloride hydrate of the present invention.

### EXAMPLES

### Example 1

A sample of crystalline anhydrous or monohydrate forms of moxifloxacin monohydrochloride, or mixtures thereof, was weighed into a beaker and placed into a controlled humidification environment at 60% relative humidity and 30° C. After 18 hours, the sample was analysed by XRPD and found to be a novel hydrate form with data illustrated in Table 1. XRPD was performed on a Brukers D8® advance diffractometer, at 30° C. between the angles of 5° and 30° 2 theta.

TABLE 1
XRPD data of novel hydrate form of moxifloxacin monohydrochloride
Angle 2 theta [°]

5.8
7.2
8.2
8.6
10.3
12.3
13.2
13.9
14.1
14.9
15.2
16.7
16.9
17.2
17.3
17.9
19.3
19.8
21.6
22.3
22.5
24.7
25.2
25.8
26.4
26.5
27.0
27.4
28.0
28.3
29.1

### Example 2

A sample of crystalline anhydrous or monohydrate forms of moxifloxacin monohydrochloride, or mixtures thereof, was weighed into a beaker and placed into a controlled humidification environment at 80% relative humidity and 30° C. After 12 hours, the sample was analysed by XRPD and found to be the novel hydrate form with XRPD data consistent with example 1 and Table 1.

The samples of the novel hydrate form prepared in examples 1 and 2 were found to be stable and exhibit no polymorphic conversion on prolonged storage over 3 weeks at ambient conditions or at high humidity accelerated conditions.

1-17. (canceled)

18. A process for the preparation of a hydrate form of moxifloxacin monohydrochloride having an X-ray diffraction pattern comprising peaks with 2 theta angles of about 5.8, 7.2, 8.6, 10.3, 17.3, 17.9, 19.3, 21.6 and 27.4 degrees when Cu Kα radiation is used, the process comprising humidification of one or more forms of moxifloxacin monohydrochloride.

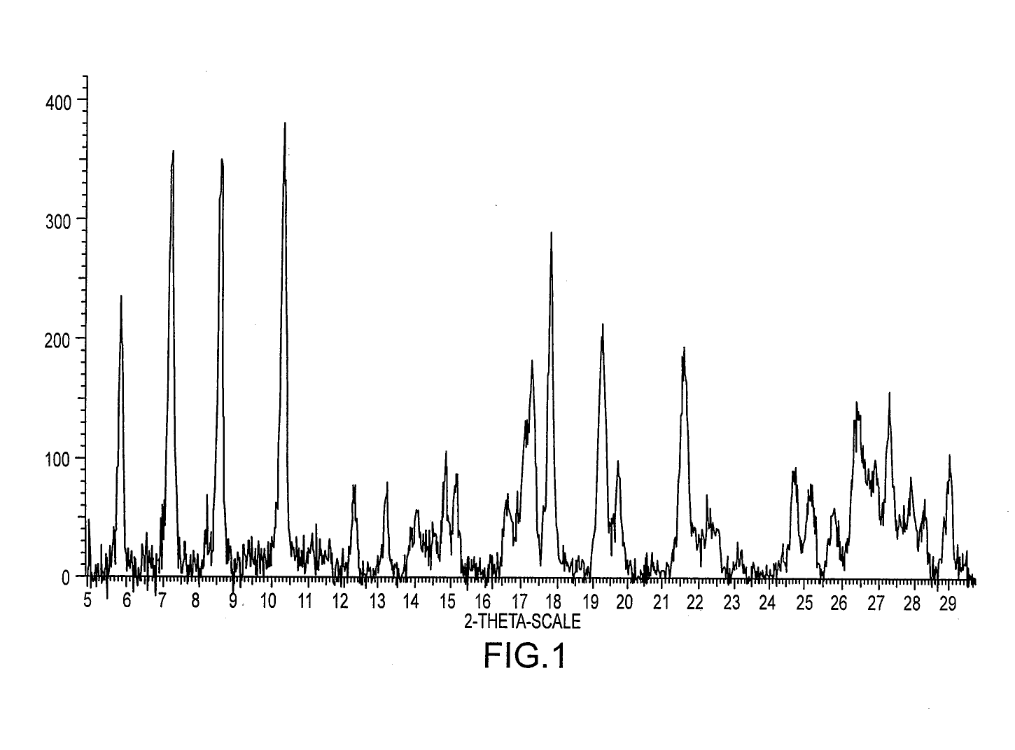

19. The process as claimed in claim 18, wherein the hydrate form of moxifloxacin monohydrochloride has an X-ray diffraction pattern substantially as shown in FIG. 1, when Cu Kα radiation is used.

20. The process as claimed in claim 18, wherein the hydrate form of moxifloxacin monohydrochloride is substantially free of other polymorphic and amorphous forms of moxifloxacin monohydrochloride.

21. The process as claimed in claim 18, wherein the humidification is carried out at 50-90% relative humidity at 25-60° C. for 8-24 hours.

22. The process as claimed in claim 21, wherein the humidification is carried out at about 60% relative humidity at about 30° C. for about 18 hours.

23. The process as claimed in claim 21, wherein the humidification is carried out at about 80% relative humidity at about 30° C. for about 12 hours.

24. A hydrate form of moxifloxacin monohydrochloride, prepared by a process as claimed in claim 18.

25. A pharmaceutical composition comprising the hydrate form of moxifloxacin monohydrochloride as claimed in claim 24.

26. The pharmaceutical composition as claimed in claim 25, wherein the composition is a solid dosage form for oral administration.

27. The pharmaceutical composition as claimed in claim 26, wherein the composition is a tablet.

28. A method of treating a bacterial or microbial infection, comprising administering a therapeutically effective amount of the hydrate form of moxifloxacin monohydrochloride as claimed in claim 24 to a patient in need thereof.

29. The method as claimed in claim 28, wherein the patient is a mammal.

30. The method as claimed in claim 29, wherein the mammal is a human.

31. A method of treating a bacterial or microbial infection, comprising administering a therapeutically effective amount of the pharmaceutical composition as claimed in claim 25 to a patient in need thereof.

32. The method as claimed in claim 31, wherein the patient is a mammal.

33. The method as claimed in claim 32, wherein the mammal is a human.
